(19) United States
(12) Patent Application Publication
(10) Pub. No.: US 2026/0053926 A1
(43) Pub. Date: Feb. 26, 2026

(54) PERSONALIZED TOPICAL FORMULATION IMPRINTED WITH QUANTUM ENERGY AND USING AI EXPERT ANALYSIS OPTIMIZATION

(71) Applicant: Luluble Inc., Mahwah, NJ (US)

(72) Inventors: Luciana de Mello Coutinho, Mahwah, NJ (US); Ai Takubo, Kailua, HI (US); Remington Kim, Paramus, NJ (US); Michele C. Duggan, Middletown, NJ (US); Leona Giat Fleissman, Ridgewood, NJ (US); Carla Mesquita D'Aprile, Sao Paoulo (BR)

(73) Assignee: Luluble Inc., Mahwah, NJ (US)

(21) Appl. No.: 18/747,463

(22) Filed: Jun. 19, 2024

Related U.S. Application Data
(60) Provisional application No. 63/521,746, filed on Jun. 19, 2023.

(51) Int. Cl. A61K 41/00; A61K 31/4166; A61K 31/5415; A61K 33/00; A61K 33/06; A61K 33/26; A61K 33/32; A61K 33/38; A61K 36/02; A61K 36/185; A61K 36/258; A61K 36/537; A61K 47/10; A61K 47/12; A61K 47/36

(52) U.S. Cl. CPC A61K 41/0004; A61K 31/4166; A61K 31/5415; A61K 33/00; A61K 33/06; A61K 33/26; A61K 33/32; A61K 33/38; A61K 36/02; A61K 36/185; A61K 36/258; A61K 36/537; A61K 47/10; A61K 47/12; A61K 47/36

(57) ABSTRACT

The present disclosure provides for a customized topical formulation energized with quantum frequencies. In at least one aspect, the topical formulation contains a base formulation and a booster formulation. In another aspect of the topical formulation, the topical formulation is created by collecting data about a user and applying artificial intelligence (AI) and optimization models to the data to arrive at the custom and/or optimized topical formulation for the user.

### CROSS-REFERENCE TO RELATED APPLICATIONS

This application claims priority under 35 USC 119(e) to U.S. Provisional Application No. 63/21,746 filed Jun. 19, 2023, the entire contents of which are incorporated by reference in its entirety.

### FIELD OF THE EMBODIMENTS

The field of the present invention and its embodiments relate to personalized topical preparations that are imprinted with electromagnetic (EM) frequencies and/or quantum energy field frequency(ies) and includes ingredients to enhance skin health and overall well-being related to EM frequency effects on the skin. In particular, the present application relates to the topical preparations for external use (e.g., skin, hair, scalp, fur, etc.) that are created via artificial intelligence (AI) and optimization modeling. The present application further describes methods of preparation of such topical preparations.

### BACKGROUND OF THE EMBODIMENTS

The skin generally acts as a protective barrier against damage, which may arise from the environment such as via chemical exposure and infectious microorganisms. Healthy skin further protects against environmental damage that may arise from contact with environmental pollutants and an excess of damaging solar radiation. As the largest single organ in the body, the skin has a multiple-layer structure, each serving separate metabolic and protective functions.

Skin is made up of three distinct layers; the epidermis, which is a thin, translucent outer layer; the dermis, which is a thicker inner layer; and a subcutaneous layer of fat. Because human skin performs several functions, including preventing excessive loss of body moisture and surface lubrication from the internal oil glands, the metabolic activity of the skin is an important factor to consider in the development of any skin care product that will be in contact with the skin, particularly in topical preparations. Several factors need to be considered, including maintaining and enhancing moisture retention and safety, because many of these products remain in contact with the skin for several hours.

In order to resolve the disadvantages mentioned herein, methods have been developed for the preparation of specific cosmetics, OTCs (over the counter drugs/medications), and medical devices created ad-hoc for the individual customer/user. For example, some methods provide for the collection of information with reference to the characteristics of the skin and to particular needs of a user and to other characteristics such as gender, age, ethnicity, etc. This information is then processed and combined to obtain the combination of ingredients for the cosmetic formulation. However, these methods produce a cosmetic based on information obtained from a questionnaire filled out by the user of the cosmetic with reference to certain needs thereof and/or to the characteristics of their skin and, therefore, do not achieve an objective and completely accurate analysis of those that are the real characteristics or requirements of the user of the cosmetic.

Thus, while there are many topical products (e.g., cosmetics) for suitable use on the skin and/or hair, there is no evidence that such products are capable of providing individualized needs to the skin and/or hair or other topically treatable area of the user, or that such products provide any particular effects for a particular user. In other words, the general populace is typically using cosmetic products created for a mass market without taking the individual user into consideration. These and other needs are addressed by the embodiments of the present application as described herein.

U.S. Patent Application Publication 2023/0029964 pertains to cosmetic formulations and compositions customized and/or personalized for rejuvenation of skin and/or hair. The cosmetic formulations are customized and/or personalized to include an extract and/or one or more molecular components in amounts and ratios naturally produced by rejuvenated cells or by cells modified toward rejuvenation. These cells are of skin and/or hair origin that have been reprogrammed, in which the skin and/or hair cells, having been rejuvenated and/or modified from the original cell, are initially obtained from a subject for which a customized cosmetic composition is created, or from a subject representative of a person for which a customized cosmetic composition is created. Reprogramming of the skin and/or hair cells provide rejuvenated cells, embryonic stem cell-like pluripotent cells, and/or trans differentiated or differentiated young or younger cells that have been trans differentiated from the rejuvenated or modified cells or differentiated from the embryonic stem cell-like pluripotent cells. Methods of selecting, preparing, and formulating the customized cosmetic products are also described.

International Application Publication WO2010/039465 pertains to a method for measuring in real-time the frequency and the power of an electromagnetic (“EM”) field emitted by a human body for different body conditions, the resonance phenomenon between the EM fields emitted by various products or electronic devices and the EM field emitted by a human body, and the signals emitted by active materials, waters, topical products, etc. A first real time spectrum analyzer is connected to a broad frequency range antenna placed in contact with the skin, electromagnetic fields emitted by a particular condition of the subject human body are measured to reveal peaks of power, and a second narrow frequency antenna is used to measure the peak more accurately. A product is applied to the condition, and a second measurement of the EM field is taken. The measurements will be particularly useful to identify skin and body deficiencies. The methods will facilitate development of treatments to mitigate/remediate skin/body deficiencies through application of topical cosmetics, OTCs, medical devices or pharmaceutical products or by the use of a device (digitized cosmetics) that emits specially selected electromagnetic signals/frequencies or their harmonics to match or interfere with the electromagnetic frequencies and their harmonics emitted by the afflicted parts of the body.

International Application Publication WO2013/113856 pertains to a process for the production of a personalized cosmetic, especially suitable for the preparation of cosmetics of the face cream, eye cream, face serum, hand cream, body cream, body oil type, and the like, with said cosmetics comprising completely natural elements of composition and of vegetal origin, defined by water, a base emulsion, active ingredients extracted from herbs and plants, one or more Bach flower essences or similar flowers, one or more essential oils, the process comprising the steps of: 1) performance of a test of bio-resonance suitable for creating an energy mapping of an individual through the detection of the frequencies of the electromagnetic oscillations emitted by the body and typical of each single individual; 2) transmission of the data detected to a device of acquisition and processing; 3) processing of the data acquired by means of software loaded on a computer or tablet, generating of a report containing an optimal combination of substances; 4) preparation of the cosmetic.

International Application Publication WO2023/008707 pertains to a smart cosmetics manager and skin care system for providing an artificial intelligence-based skin care solution, comprising: a smart cosmetics manager, which stores and manages a plurality of cosmetics, transmits, to a solution server, a user's facial image and identification information and storage status information about the plurality of cosmetics, and receives customized skin care solution information from the solution server and displays same; a user terminal for transmitting user information to the solution server, and retrieving the customized skin care solution information; and the solution server, which generates the customized skin care solution information on the basis of the user information, the identification information and the user's facial image, so as to provide the same to the smart cosmetics manager or the user terminal, wherein the user information and pieces of information related to facial skin and the stored cosmetics are received from the smart cosmetics manager and the user terminal, and the received pieces of information are analyzed on the basis of artificial intelligence so as to provide a user-customized skin care solution, and thus useful information enabling a user's skin to be maintained in the best condition without a separate consultation or seeing a dermatologist is provided, the cosmetics or makeup items can be stored in an optimal state, and space utilization can be maximized.

As noted, various topical cosmetics are known in the art. However, their structure and means of operation are substantially different from that of the present disclosure. The present invention and its embodiments seek to develop personalized topical preparations/products that are tailored to each user. At least one embodiment of this invention is presented in the drawings below and will be described in more detail herein.

Native and Non-Native Physics, Biology, and Biofield table/definitions describing the processes used by nature and manufactured by humans are listed below:

### Biology (Native/Non-Native) Table

Biology: Native/Non-Native

| Item/ | | |
|---|---|---|
| Biological | | |
| Structure | Native | Non-Native |
| Melanopsin | Stimulated by energy (EM & | Botanical with retinoid |
| | thermal) | which would convert |
| | Protects against oxidative stress | to retinal |
| | & inflammation | (chromophore retinal |
| | Increases UVR dependent Ca++ | in opsins) |
| | mobilization (electron transport | Frequency used to |
| | channels) | improve cell health |
| | Protects against DNA damage (I | this allows OPN4 to |
| | think - need full article) | function in a positive |
| | Opsins-UVR and VL induced | way: apoptosis for |
| | pigmentation | damaged cells, |
| | Opsins-Keratinocyte | enhance electron |
| | differentiation | channels in cells, |
| | opsins-UVR-induced photoaging | protect DNA histones |
| | Apotosis | |
| | Opsins-Modulation of circadian | |
| | clock genes | |
| | Opsins-Hair follicle growth | |
| Melanin | Stimulated by EM (ELF - 50- | Use ELF/LF - |
| | 75 hz) | imprinting |
| | Antioxidant (as well as | L-Tyr, L-DOPA |
| | giving rise to ROS's) | starting materials for |
| | | melanogenesis |
| Blood | Nitric oxide increase due to | L-Arginine |
| Vessels | opsins and blue light dilate blood | 60 hz to upregulate |
| | vessels and lower blood pressure | NOS (nitric oxide |
| | | synthase) |
| Proteins | Lower oxidative stress protects | Beta-carotene (to |
| | all proteins and the skin | protect against ROS |
| | | damage to proteins) |
| Mitochondria | UV irradiation and IBMX-as well | Melatonin (AO and |
| | as melanogenin-may influence | promotes AO enzyme |
| | stress response pathways, which | production by |
| | can alter the levels of tyrosinase | mitochondria) |
| | | Melatonin can be |
| | | synthesized by the |
| | | mitochondria and also |
| | | can be transported |
| | | into the mitochondria |
| | | from the cell's |
| | | cytoplasm. |
| | | Melatonin can |
| | | penetrate the skin |
| Melatonin | Produced in mitochondria as well | L-Tryptophan - |
| | as in the pineal gland | starting material in |
| | Acts in mitochondria as an | melatonin synthetic |
| | antioxidant and upregulates | pathway |
| | antioxidant enzymes in the | |
| | mitochondria | |

### Physics (Native/Non Native) Table

PHYSICS − SUN + EARTH

| Native | Non Native |
|---|---|
| Electromagnetic | Frequency generators |
| Wavelengths | Biomodulators |
| Frequencies | |

V = λ*c
V = frequency (Hz) (1/s)
λ = wavelength (m)
c = speed of light · 10\hat{ }2 (m/s)

| | | |
|---|---|---|
| Carrier | Waters: | |
| | Glacial | Electro-Magnetic |
| | Mineral/Spring | Structures |
| | Volcanic | |
| Enhancers | Plants | Silicon-rich extracts |
| | Dirt | Botanical extracts |
| | Crystals | Essential Amino Acids |
| | Salts | Frequency rich modulators |
| | Thunder | |
| Product | Walk in Nature | Quantum enhanced |
| | without sunscreen | topical |
| | Walk in Nature | products |
| | without sunglasses | |
| | Grounding | |
| | Organic food | |
| | Sleep | |
| Efficacy | Quantum/Scalar Energy | Scalar/Orgone Generators |

The Biofield or biological field is a complex organizing energy field engaged in the generation, maintenance, and regulation of biological homeodynamics, which is a useful concept that provides the rudiments of a scientific foundation for energy medicine and thereby advances the research and practice of it. (“Biofield Science and Healing: History, Terminology, and Concepts”, Rubik et al. Global Adv Health Med., 2015 4 (Supp), 8-14.). The human Biofield is about 5-6 ft. It can also be called Scalar, Aura, Chi, Akasha, Ether. This area is where emotions are housed. (“Scalar Energy: The Essence of Life”)

Skin emits Biophotons and ultra-weak photon emission (UPE) measurements can be used to help us better understand the interaction between skin and the outside world. (“The application of ultra-weak photon emission in dermatology”)

Redox potential, also known as oxidation-reduction potential (ORP), is a measure of the tendency of a chemical species to acquire electrons and thereby be reduced or to lose electrons and be oxidized. A higher ORP represents a greater oxidizing ability, while a lower ORP indicates a greater reducing ability. Redox reactions in aqueous solutions are significantly important in biological and environmental systems. They support and maintain life by gathering and dissipating energy to generate and propagate low-entropy living systems.

### SUMMARY OF THE INVENTION

It is an objective of this invention and its embodiments to provide through the personalized topical imprinted preparation/products tailored to each user a negatively charged product that enhances the overall functioning of the organism/body. The negatively charged preparation/product will increase the body's/organism's ability to produce new cells that are able to improve and maintain overall health while eliminating poorly functioning cells and toxins from the body/organism. The recipient of this negative charge in an embodiment is the melanin present in the skin, which acts as a super-conductor of energy throughout the body, promoting the flow of energy imprinted on the preparation/product and fostering the interaction between the skin, brain, and gut.

The interaction between the gut and the brain is widely discussed in scientific circles, while the relationship between the skin, gut, and brain axis is still being explored. In an embodiment, this invention aims to clarify the missing link between the skin-gut-brain connection. There exist scientific studies that suggest that melanin present in various body parts, such as skin and hair (peripheral melanin) and the brain (neuromelanin), protects the body against reactive oxygen species (ROS). This protective property depends on both the redox potential and the charge on the radical. Reactive oxygen species are byproducts of cellular processes. When ROS accumulate in cells, they can cause stress, premature aging, and health issues. Melanin scavenges ROS, boosting antioxidants and eliminating free radicals. Studies suggest that melanin may support the immune system and reduce inflammation in the body. Without being bound by theory, melanin acts as an excellent semiconductor and, in the presence of UV light, uses water, whose components such as oxygen can serve as a terminal electron acceptor in the electron transport chain. Melanin also delivers oxygen to cells and mitochondria and also provides energy to cells, particularly in the substantia nigra, a key communication hub in the brain. Additionally, melanin can recreate water and sequester heavy metals and pollutants. It is constantly renewed in the body during sleep and functions as a biochemical neurotransmitter. Melanin is an exceptional charge separator, creating more free electrons wherever it resides in the body. This increases the bioavailability of electrons to cells, thereby enhancing cellular redox.

Electromagnetic fields are divided as below: (“Electric & Magnetic Fields”).

| Radiation | | Forms of | Source |
|---|---|---|---|
| Type | Definition | Radiation | Examples |
| Non-Ionizing | Low to mid-frequency radiation which is | Extremely | microwave ovens |
| | generally perceived as harmless due to | low | computers |
| | its lack of potency | frequency | house energy smart |
| | | (ELF) | meters |
| | | radio | wireless (Wi-Fi) networks |
| | | frequency | cell phones |
| | | (RF) | Bluetooth devices |
| | | microwaves | power lines |
| | | visual light | MRIs |
| Ionizing | Mid to high frequency radiation which | ultraviolet | sunlight |
| | can, under certain circumstances, lead | X-rays | X-rays |
| | to cellular and/or DNA damage with | Gamma | some Gamma rays |
| | prolonged exposure | | |

Electric and magnetic fields (EMFs) are invisible to humans, and are often referred to as radiation. These fields are associated with the use of electrical power and various forms of natural and man-made lighting. EMFs are typically grouped into one of two categories by their frequency: Non-ionizing radiation or low-level radiation, which is generally perceived as harmless to humans and ionizing radiation, which is a high-level radiation, which has the potential for cellular and/or DNA damage.

There are potential beneficial health effects of exposure to certain electromagnetic fields, specifically ELF-EMFs (extremely low energy electric and magnetic fields) (“Capone F, Dileone M, Profice P, Pilato F, Musumeci G, Minicuci G, Ranieri F, Cadossi R, Setti S, Tonali PA, Di Lazzaro V. Does exposure to extremely low frequency magnetic fields produce functional changes in human brain? J Neural Transm (Vienna). 2009 March”) and certain radio frequencies called Fractional-Radio frequencies (FRs) (“@inproceedings {Lee2014ConsensusRO, title={Consensus Recommendations on the Use of a Fractional Radiofrequency Microneedle and Its Applications in Dermatologic Laser Surgery}, author={Seung Jae Lee and Un Cheol Yeo and Seong Ho Wee and Jae Hong Shim and”) and those benefits in embodiments of the present invention are exploited.

Thus, the present invention and its embodiments provide for personalized topical imprinted preparations/products tailored to each user to: 1) enhance the overall functionality of the formula through the use of quantum physics energy via imprinting extremely low frequencies (ELFs) (and/or) fractional radio frequencies (FRs); 2) rebalance the state of well-being of humans and animals by utilizing neuro-cutaneous interactions based on physics, biology, and consciousness guidelines; 3) target the neuro-cutaneous interactions to enhance the appearance of the skin/hair/scalp/fur; 4) implement an artificial intelligence (AI) algorithm to meet the needs of individual users; and 5) support via chemical reactions measurements that involve the transfer of electrons from one species to another (such as but not limited to redox measurements, single electron transfer, and/or biological/bio-resonance measurements (such as, but not limited to GDV (gastric dilation volvulus), biofeedback, GSR (galvanic skin response), HRV (heart rate variability), EEG (electroencephalogram), ECG (electrocardiogram), blood oxygen) in order to demonstrate formula efficacy for the user.

In at least one embodiment, the topical preparation is imprinted with EM frequency(ies) (or) another quantum energy field frequency.

The topical preparation may also include a topical base formulation that includes a pH adjusting agent, a chelating agent, at least one emulsifier, at least one emollient, an antimicrobial agent, an antioxidant, and/or an electrolyte.

In yet another aspect of the embodiments, the topical preparation includes about 90% to about 99.9% of a topical base preparation, and about 0.1% to about 10% of a companion preparation in which at least one of the formulations is imprinted with EM frequency(ies) (and/or) another quantum energy field frequency.

In yet another aspect of the embodiments, there is a method of preparing a topical preparation, the method includes the steps of: collecting one or more variables about a user of the topical preparation to generate a data set, administering, via a processor, at least one artificial intelligence (AI) model to the data set to generate an AI driven data set, administering, via a processor, at least one optimization engine to the AI driven data set to generate an optimized data set generating, via a processor, the topical preparation based on the optimized data set.

The method may also include the step of applying a pulsed electromagnetic field, remote quantum entanglement energy, plasma energy, and/or scalar energy to the topical preparation for at least one period of time. Other technical features may be readily apparent to one skilled in the art from the figures, descriptions, and/or claims.

The method may also include embodiments where the pulsed electromagnetic field, remote quantum entanglement energy, plasma energy, and/or scalar energy is applied to the topical preparation before use by the end user.

The method may also include a frequency of the pulsed electromagnetic field, remote quantum entanglement energy, plasma energy, or scalar energy in the range of 1 to 3000 Hertz or in an embodiment, less. Other technical features may be readily apparent to one skilled in the art from the description and claims.

The method may also include materials to be used by the skin to enhance the effect of the imprinted information, such as L-tyrosine, L-tryptophan, L-arginine, inulin, allantoin, diatomaceous earth, bentonite clay, algae, Chlorella, coenzyme Q-10, xylitol, retinoids, St. John's wort extract, horsetail extract, hematite extracts, crystal extracts, hyaluronic acid, sodium hyaluronate, methylene blue, silver, gold, copper, and/or bronze.

In general, the present invention and its embodiments succeed in conferring the benefits and objectives as disclosed herein.

It is an object of the present invention and its embodiments to provide a topical formulation that is customized for the individual user.

It is an object of the present invention and its embodiments to provide a topical formulation that is created with the implementation of an AI model.

It is an object of the present invention and its embodiments to provide a topical formulation that has a particular effect on a user when used as recommended.

It is an object of the present invention and its embodiments to provide a topical formulation that is imprinted with a pulsed electromagnetic field, remote quantum entanglement energy, plasma energy, and/or scalar energy.

It is an object of the present invention and its embodiments to provide a topical formulation that is stable.

It is an object of the present invention and its embodiments to provide a topical formulation capable of being monitored by a user for effectiveness by measuring bio-resonance variables associated with the user.

It is an object of the present invention and its embodiments to provide a topical formulation that takes advantage of neuro-cutaneous interactions.

It is an object of the present invention and its embodiments to provide a topical formulation that strives to enhance the appearance of the skin/hair/scalp/fur.

Further objects of the present invention and its embodiments may be readily apparent to one of ordinary skill in the art from the following descriptions and claims.

### BRIEF DESCRIPTION OF THE SEVERAL VIEWS OF THE DRAWINGS

FIG. 1 shows various devices that irradiate in different regions of the electromagnetic spectrum as well as a depiction of the relative frequencies of each type of irradiation.

### DETAILED DESCRIPTION OF THE EMBODIMENTS

The preferred embodiments of the present invention will now be described with reference to the drawings. Identical elements in the various figures are identified with the same reference numerals.

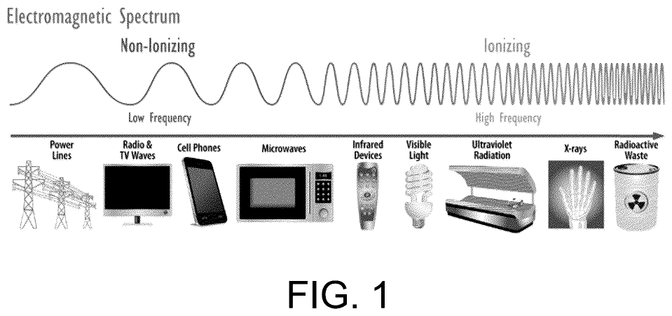

As is shown in FIG. 1, different wavelengths of the electromagnetic spectrum cause different chemical transitions, and the devices that are used to cause those various transitions are shown in FIG. 1.

Reference will now be made in detail to each embodiment of the present invention. Such embodiments are provided by way of explanation of the present invention, which is not intended to be limited thereto. In fact, those of ordinary skill in the art will appreciate upon reading the present specification and viewing the present drawings that various modifications and variations can be made thereto.

When introducing elements of the present disclosure or the embodiments thereof, the articles “a,” “an,” and “the” are intended to mean that there are one or more of the elements. Similarly, the adjective “another,” when used to introduce an element, is intended to mean one or more elements. The terms “including” and “having” are intended to be inclusive such that there may be additional elements other than the listed elements.

All percentages and ratios used herein are by weight of the total composition and all measurements made are at room temperature unless otherwise specified. All percentages, parts, and ratios are based upon the total weight of the compositions disclosed herein, unless otherwise specified.

The term “carrier” as used herein refers to organic or inorganic ingredients, natural or synthetic, with which an active ingredient is combined to facilitate the application of a composition. In the present context, the terms “carrier” and “vehicle” may be interchangeably used. The term “carrier” includes, but is not limited to, water, alone or in combination with other materials.

The term “cream” as used herein refers to viscous liquids or semisolid emulsions, either oil-in-water or water-in-oil.

The terms “comprising,” “including,” “containing” and the like as used herein are inclusive or open-ended terms that do not exclude additional, un-recited elements or method steps.

To the extent used herein, the phrase “consisting of” and grammatical equivalents thereof exclude any element, step, or ingredient not specified in the claim.

To the extent used herein, the phrase “consisting essentially of” limits the scope of a claim to the specified materials or steps and those that do not materially affect the basic characteristic or characteristics of the claimed disclosure.

The term “emollient” as used herein refers to an agent that softens and smooths the skin and/or hair and/or scalp and/or fur. Emollients are also occlusive agents, e.g., substances that provide a layer of protection that helps prevent moisture (water) loss from the skin and can also be a barrier from/to foreign agents. An emollient may be, for example, selected from the group consisting of fats, oils, fatty alcohols, fatty acids, fatty acid ethers and fatty acid esters and/or mixtures thereof. Several emollients may be present in a single composition, selected for their individual properties and blended to achieve a desired result.

The term “humectant” as used herein refers to a substance that bonds to water molecules to increase the water content in the skin itself. Many humectants also have emollient properties, although not all emollients are humectants. Several humectants may be present in a single composition, selected for their individual properties and blended to achieve a desirable result.

The term “lotion” as used herein refers to preparations to be applied to the skin surface without friction and are typically liquid or semiliquid preparation in which solid particles, including the active agents, are present in a water or alcohol base.

The term “ointment” as used herein refers to a semisolid preparation containing an ointment base and optionally one or more active agents.

The term “preservative” as used herein refers to a natural or synthetic chemical that is added to products to prevent decomposition by microbial growth or by undesirable chemical changes.

The terms “patient,” “subject,” “user,” or “host” as used herein may mean either a human or non-human animal, such as primates, mammals, and vertebrates. In an embodiment, a “subject” is a human.

The term “topical administration” as used herein means delivery of an active agent to the skin or hair.

The term “total daily dose” as used herein refers to the total amount of compound or composition administered to the patient in one 24-hour period, regardless of whether the protocol calls for a once-daily, twice-daily, or thrice-daily administration of the compound or composition. Thus, the total amount of compound or composition is summed for a given 24-hour period to determine how much total compound or composition the subject is to be administered in a given day.

The compositions or formulations of the present invention and its embodiments can be administered topically to a subject, e.g., by the direct laying on or spreading of the composition on the epidermal or epithelial tissue of the subject, or transdermally via a “patch”. The formulation may be in the form of an ointment, cream, lotion, gel, paste, a solid “stick”, foam, solution, balms, sprays, suspensions, ointments, films or the like, that can be applied to the skin by hand or applicator, for example, by rubbing or spraying.

In exemplary embodiments, the carrier is in the form of a cream, lotion, serum, gel, hydrosol, ointment, surfactant systems, mask, wax-based systems, mascara, lubricants, or powder.

Compositions or formulations of the present invention may be beneficial in preventing, treating, managing, and/or ameliorating a variety of skin/scalp and/or hair/fur conditions. Specifically, the compositions may be beneficial in preventing, treating, managing, ameliorating and/or reducing the incidence or severity of at least one of: wrinkles, fine lines, blemishes, skin discoloration, dry skin, skin irritation, saggy skin, inelastic skin, enlarged pores, acne, acne scars, inflammation, crow's feet, laugh lines, drooping eyelids, frown lines, dullness, dull skin tone, dark circles under the eyes, itchy skin, hyperpigmentation, uneven skin tone, tautness and/or collagen loss. In addition, the topical preparation may be beneficial for biological and/or environmental conditions such as or related to sleep deprivation, dehydration, high sun exposure, bad nutrition, alcohol consumption, smoking, pollution, hard water, arid climate-humid climate, frigid climate, hot climate, seasonal allergies, stress, fatigue, depression, radiance, sagging, firmness and under eye bags. More specifically, the compositions may be beneficial to prevent or reduce signs of aging, cleanse the skin, remove excess oil, reduce the size and redness of acne scars, renew skin, normalize skin, exfoliate the skin, provide smooth & soft skin texture, invigorate skin by lifting away dead skin cells, clear skin, reduce appearance of pores, help balance skin's pH level, provide clearer complexion, and/or provide softer & smoother skin. The compositions may also support a sense of overall well-being since skin is part of the peripheral endocrine system.

The compositions of the present invention can further comprise one or more additional pharmaceutically acceptable excipients, such as one or more thickening agents, inert carriers, stabilizers, chelating agents, buffers, moisture absorbents, aesthetic modifiers, antimicrobials, fragrances, or colorants. Various enhancements may be made to the composition to improve odor, scent, or aroma of the topical composition.

The pH of the aqueous phase can be adjusted as needed. Agents suitable for adjusting the pH include, but are not limited to arginine, NaOH, triethanolamine, monoethanolamine, HCl, acetic acid, citric acid, phosphoric acid, lactic acid or glycolic acid. Typically, the one or more pH adjustment agents are present in an amount from about 0.01 to about 5% by weight of the total composition. In embodiments, the pH adjustment agents are present in an amount from about 0.1 to about 1.0% by weight of the total composition.

Suitable humectants may include, but are not limited to polyhydric alcohols including glycerin, diglycerin, triglycerin, polyglycerin, polypropylene glycol, polyethylene glycol, ethylene glycol, diethylene glycol, triethylene glycol, propylene glycol, dipropylene glycol, hexylene glycol, 1,3-butylene glycol, 1,4-butylene glycol, ethylene glycol monoalkyl ether, diethylene glycol monoalkyl ether, glucose, maltose, sucrose, lactose, xylitose, xylitol, sorbitol, mannitol, maltitol, panthenol, pentaerythritol, lanolin, and hyaluronic acid and its salts. It should, of course, be understood that combinations of two or more humectants can be included in the present compositions. Typically, the one or more humectants are present in an amount from about 1% to about 20% by weight of the total composition. In embodiments, humectants are present in an amount from about 2 to about 5% by weight of the total composition. In embodiments, a combination of humectants is present, with each individual humectant being present in an amount from about 0.5% to about 5% or 2% to 4% or 1% to 5% by weight of the composition.

Representative, non-limiting examples of suitable preservatives may include caprylyl glycol, sorbic acid, benzoic acid, methyl-paraben, propyl-paraben, methylchloroisothiazolinone, methylisothiazolinone, diazolidinyl urea, chlorobutanol, triclosan, benzethonium chloride, p-hydroxybenzoate, chlorhexidine, digluconate, hexadecyltrimethylammonium bromide, alcohols, benzalkonium chloride, boric acid, bronopol, butylparaben, butylene calcium acetate, calcium chloride, calcium lactate, carbon dioxide, bentonite, cetrimide, cetylpyridinium chloride, chlorhexidine, chlorobutanol, chlorocresol, chloroxylenol, citric acid monohydrate, cresol, dimethyl ether, ethylparaben, hexetidine, imidurea, isopropyl alcohol, lactic acid, monothioglycerol, pentetic acid, phenol, phenoxyethanol, phenylethyl alcohol, phenylmercuric acetate, phenylmercuric borate, phenylmercuric nitrate, potassium benzoate, potassium metabisulfite, potassium sorbate, propionic acid, propyl gallate, propylene glycol, sodium acetate, sodium benzoate, sodium borate, sodium lactate, sodium sulfite, sodium propionate, sodium metabisulfite, xylitol, sulfur dioxide, carbon dioxide, and/or any combination thereof.

Representative, non-limiting examples of antioxidants may include synthetic antioxidants such as BHT (butylated hydroxytoluene), BHA (butylated hydroxyanisole), and TBHQ (tertiary butyl hydroquinone) or mixtures thereof. Other antioxidants suitable for use include acetyl cysteine, ascorbic acid polypeptide, ascorbyl dipalmitate, ascorbyl methylsilanol pectinate, ascorbyl palmitate, ascorbyl stearate, Cocos nucifera liquid endosperm, Cocos nucifera juice, cysteine, cysteine HCl, diamyl hydroquinone, di-t-butylhydroquinone, dicetyl thiodipropionate, dioleyl tocopheryl methylsilanol, disodium ascorbyl sulfate, distearyl thiodipropionate, ditridecyl thiodipropionate, dodecyl gallate, erythorbic acid, esters of ascorbic acid, ethyl ferulate, ferulic acid, gallic acid esters, glycerin, hydroquinone, isooctyl thioglycolate, kojic acid, magnesium ascorbate, magnesium ascorbyl phosphate, methylsilanol ascorbate, natural botanical antioxidants such as green tea or grape seed extracts, nordihydroguaiaretic acid, octyl gallate, phenyl thioglycolic acid, potassium ascorbyl tocopheryl phosphate, potassium sulfite, propyl gallate, quinones, rosmarinic acid, sodium ascorbate, sodium bisulfite, sodium erythorbate, sodium metabisulfite, sodium sulfite, superoxide dismutase, sodium thioglycolate, sorbityl furfural, thiodiglycol, thiodiglycol amide, thiodiglycolic acid, thioglycolic acid, thiolactic acid, thiosalicylic acid, tocophereth-5, tocophereth-10, tocophereth-12, tocophereth-18, tocophereth-50, tocopherol, tocophersolan, tocopheryl acetate, tocopheryl linoleate, tocopheryl nicotinate, tocopheryl succinate, and/or tris(nonylphenyl)phosphite and mixtures thereof.

Water may be present in the composition of the present invention. In exemplary embodiments, water is present in an amount of about 20% to about 95% by weight of the composition, or any range and/or individual value therein, such as, but not limited to, about 55% to about 75% or about 60% to about 70% by weight of the composition. In certain embodiments, water is present in a first composition of the present invention in an amount of about 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, or 79% by weight of the composition or any range and/or individual value therein.

In at least one embodiment, the topical base formulation comprises any of the following or mixtures thereof:

| Function | Ingredient |
|---|---|
| Vehicle | Water (Aqua) |
| | Hydrosol, aloe juice |
| | Citric Acid |
| | Acetic Acid, Glycolic Acid, Lactic Acid |
| | Arginine |
| pH adjuster | Sodium Hydroxide, TEA |
| | Sodium Phytate |
| | EDTA |
| Chelating Agent | Humic/Fulvic Acids |
| Cofactor for enzymatic reaction | Calcium Chloride |
| | NaCl, KCl, Magnesium Chloride, Magnesium |
| | Sulfate, Sodium Chloride, Sea Salt |
| | Sodium Bicarbonate |
| | Calcium Chloride |
| | NaCl, KCl, Magnesium Chloride, Magnesium |
| | Sulfate, Sodium Chloride, Sea Salt, Sodium |
| Electrolyte | Bicarbonate |
| | Lysolecithin (and) Sclerotium gum (and) Xanthan |
| | gum (and) Pullulan |
| | natural gums (i.e. xanthan, HEC), clays |
| Structuring agent | (veegum, bentonite, smectite), carbomers |
| | Xylitol, Sodium PCA, |
| Skin-Conditioning Agent | polyhydric alcohols (glycols), lubragels |
| | Cocos Nucifera (Coconut) Liquid Endosperm |
| | (and) Glycerin (and) Cocos Nucifera (Coconut) |
| | Fruit Juice |
| | antioxidants: BHT, vitamin E and derivatives, |
| Antioxidant/electrolyte | Astaxanthin |
| | Glycerin |
| | polyhydric alcohols (glycols), lubragels, sugar, |
| Humectant | polyglyceryl esters |
| | Inulin |
| prebiotic/aesthetics modifier | Polymnia Sonchifolia Root Juice, Yogurt |
| | Glyceryl Stearate Citrate |
| Emulsifier | non-ionic, anionic, cationic(s) |
| | Glyceryl Stearate |
| Emulsifier | non-ionic, anionic, cationic(s) |
| | Cetearyl Olivate (and) Sorbitan Olivate |
| Emulsifier | non-ionic, anionic, cationic(s) |
| | Glyceryl Stearate (and) Cetearyl Alcohol (and) |
| | Sodium Olivate (and) Inulin |
| Emulsifier | non-ionic, anionic, cationic(s) |
| | Cocos nucifera (Coconut) oil |
| | hydrocarbons, fatty esters, vegetable and animal |
| Emollient | oils, butters, waxes, silicones, fatty alcohols |
| | Coenochloris Signiensis Extract (and) |
| | Maltodextrin (and) Lecithin (and) Aqua |
| Promotables | collagen, herbal extracts, |
| | Hydrogenated Olive Oil (and) Olea Europaea |
| | (Olive) Fruit Oil (and) Olea Europaea (Olive) Oil |
| | Unsaponifiables |
| | hydrocarbons, fatty esters, vegetable and animal |
| Emollient | oils, butters, waxes, silicones, fatty alcohols |
| Emollient | Butyrospermum Parkii (Shea) Butter |
| | hydrocarbons, fatty esters, vegetable and animal |
| | oils, butters, waxes, silicones, fatty alcohols |
| | Oryza Sativa (Rice) Bran Wax |
| | body melting waxes, powders such as nylon |
| | powder, silica beads, PMMA, corn starch, boron |
| Aesthetics modifier | nitride |
| | Caprylyl Glycol |
| | Phenoxyethanol, Parabens, Benzoic Acid, |
| Antimicrobial Agent, Skin- | Potassium Sorbate, Sodium Benzoate, tea tree |
| Conditioning Agent | oil |
| Oil Absorbing agents | silica |
| astringents | ethanol, isopropanol, witch hazel |
| | Nicotinamide Mononucleotide, peptides, CBD |
| | Hematite, Ginseng, Sodium Hyaluronate, St. |
| | John's Wort, Xylitol, Methylene Blue, |
| | Diatomaceous Earth, Coenzyme Q-10, Bentonite |
| | Clay, Algae, Allantoin, Inulin, Melatonin, Melanin, |
| | Tyrosine, Sodium Bicarbonate, Sodium Chloride, |
| | Calcium Chloride, Magnesium Bicarbonate, |
| Electron/Photon enhancers | Magnesium Sulfate, Sucrose |

In addition to the above topical base formulation, there is also alternatively and/or additionally a booster formulation that is combined with the topical base preparation to arrive at the present topical formulation of the present application. The constituents of the booster formulation may comprise anti-wrinkle, anti-greying, soothing, complexion-enhancing, moisturizing, repairing, firming, anti-inflammatory, hydrating, UV protecting, anti-stress, and/or slimming agents.

The following examples illustrate three different formulations utilizing the topical base formulation described herein along with the booster formulation in varying amounts (e.g., 1×, 2×, etc.). The representative examples below are illustrative and the actual amounts in the composition may vary.

| Example #1 with | | Example #2 with | Example #3 with |
|---|---|---|---|
| single booster | | double booster | single booster |
| formulation | | formulation | formulation |
| topical base formulation | 94.00 | 94.00 | 93.00 |
| booster formulation | 3.00 | 4.50 | 6.00 |
| q.s. water | 3.00 | 1.50 | 1.00 |
| total | 100% | 100% | 100% |

In order to make the topical formulations as described herein, there is, at a high level, at least a two-step process to be followed: 1.) implementing, via a processor, an artificial intelligence (AI) model on a computing device; and 2.) implementing, via a processor, an optimization algorithm on a computing device.

First, the user's individual needs are preferably captured through data inputs by the consumer self-assessing and/or testing results and/or photographs of skin conditions. This may be done electronically or otherwise. In some embodiments, photographs are taken and uploaded to a computing device. In other embodiments, the computing device running the AI model and the optimization algorithm, serves to directly capture the representation of the skin and/or hair of the user. Further, the user's emotional condition and biological energy state are included in the process via a questionnaire.

The AI model (e.g., expert system, neural network, etc.) will preferably assign a ranking to each potential ingredient with respect to at least each of skin, wellness, and environmental condition of an intended user. A higher score means that the ingredient is more beneficial for the condition and the user, and a lower score means that the ingredient is harmful for the condition and the user. The output of the AI model based on this data is then fed into a specialized optimization algorithm, which outputs the optimal combination of ingredients that is presented to the user. It is intended that this algorithmic process processes all ingredients, conditions, and rankings to optimize and find a combination of ingredients that results in the highest possible combined net ranking thereby providing the most advantageous effects to the user.

In an embodiment, the AI system of the present invention can use any of a plurality of learning algorithms. In a variation, the learning algorithm (or prediction models) may be one or more of a linear regression algorithm, a logistic regression algorithm, a decision tree algorithm, SVM algorithm, Naïve Bayes algorithm, KNN algorithm, K-means algorithm, Random Forest algorithm, support-vector machine algorithm, gradient boosting algorithm, DBSCAN algorithm, dimensionally reduction algorithm, gradient boosting algorithm, and/or an AdaBoosting algorithm, or combinations thereof. In a variation, the model may involve supervised learning, unsupervised learning, semi-supervised learning, a deep learning algorithm, reinforcement learning, a regression method, an instance-based method, a decision tree learning method, a Bayesian method, a kernel method, a clustering method, an associated rule learning algorithm, an artificial neural network model, a dimensionality reduction method, an ensemble method, and/or other suitable AI approaches.

In an embodiment, the AI may use learning to generate updated data wherein combinations of ingredients may be evaluated to see if they have any effect on each other and the optimization therefore improved over time. That is, the more data that is obtained, a formulation combination (and recommendations on the ingredients) can be constantly improved (based upon the data).

The methodology may further leverage AI using an expert system and support vector machines. This allows for the AI model to analyze the ingredients (or potential ingredients) and the interactions between neurotransmitters as well as the conditions said neurotransmitters are associated with to optimize and generate a combination of ingredients for the topical formulation that will provide a desired effect.

In a preferred embodiment, the topical formulation contains a combination of between about 10 and 15 of the most desirable functional ingredients to generate an individualized formula. In another embodiment, the topical formulation contains a combination of between 5 and 10 of the most desirable functional ingredients to generate an individualized formula. In yet a further embodiment, the topical formulation contains a combination of between 1 and 3 of the most desirable functional ingredients to generate an individualized formula. However, regardless of the number of functional ingredients in the topical formulation, the topical formulation preferably contains the optimal combination of functional ingredients specific to each user generated by the AI model and optimization engine.

Once the product composition has been determined by the AI model and optimization engine, it can be completed utilizing known methods. Once completed, however, the present invention calls for one or more low electromagnetic frequencies commensurate with known frequencies that are beneficial to the human physiology (e.g. 432 Hz, 528 Hz) delivered via frequency generator (e.g., TrueRife System, Spooky2) to the product during batching and/or filling for a predetermined amount of time. The “energized” product is intended to provide a heightened state of well-being by delivering quantum field energy (e.g. PEMF (pulsed electromagnetic field), TENS (transcutaneous electrical nerve stimulation), plasma, scalar, remote entanglement, etc.) within the formulation circumventing the need to apply these frequencies directly to the skin. It is further believed, without being bound by theory, that quantum field energy imprinted topical formulation (“energized” formulation) will be more stable due to the addition of this additional energy. In at least one embodiment, the shelf life of the product will be extended using packaging and an EMF-blocking process to ensure that the imprinted frequency will be maintained for the intended shelf-life of the product.

Listed below are the processes used and preliminary testing results demonstrating the frequency imprinting process and its enhanced shelf life

| Process | Explanation | Condition |
|---|---|---|
| STEP 1 | molecular structuring process | A @20-40° C. |
| | | B @70-80° C. |
| STEP 2 | 528 Hz frequency imprinting utilizing | A @20-40° C. for 120 |
| | PEMF coil | min |
| | | B @70-80° C. for 120 |
| | | min |
| | | C @20-40° C. for 10 |
| | | min |
| | | D @20-40° C. for 20 |
| | | min |
| | | E @20-40° C. for 30 |
| | | min |
| STEP 3 | frequency/structure shelf-life | room temperature |
| | extension process | |

Formula #1 testing results:

| | | Description | | | | | | | | |
|---|---|---|---|---|---|---|---|---|---|---|
| | | | | | STEP 1A | STEP 1A | | STEP 1A | STEP 1A | STEP 1A |
| | | NO | STEP 1A | STEP 1B | STEP 2A | STEP 2B | STEP 1A | STEP 2C | STEP 2D | STEP 2E |
| | | STEPS | STEP 3 | STEP 3 | STEP 3 | STEP 3 | STEP 2A | STEP 3 | STEP 3 | STEP 3 |
| | | Sample IDs | | | | | | | | |
| | | G1/I1 | H1A | H1B | H2 | H4 | H3 | I4 | I5 | I6 |
| OPR | Day 0 | 68 | 40 | 40 | 36 | | | 51 | 48 | 46 |
| measure- | Day 2 | | 40 | 11.9 | 15 | 11 | 16 | | | |
| ments | Day 14 | 65 | 58 | 68 | 57 | 52 | 59 | 68 | 67 | 72 |
| in mV | Day 28 | | 60 | 68 | 54 | 53 | 58 | | | |
| | Day 86 | 72 | | | | | | | | |
| | Day 110 | | 59 | 60 | 62 | 68 | 75 | | | |
| | Day 113 | 75 | | | | | | | | |

Formula #1: DI Water, Magnesium Sulfate @1%, Calcium Chloride@0.1%, Sodium Bicarbonate@0.1%
Samples G1/I1 show high ORP values and a slight increase in ORP values from Day 0 to Day 113 due to aging.

Samples H1A and H1B that were processed using STEP1 and STEP3 (therefore no frequency imprinting) show a decrease in ORP values compared to samples G1/11. It demonstrates the ability of STEP 1 to successfully structure the molecule and STEP 3 to protect the extension of the shelf life of the molecular structure of the formula. It is noted that an increase in temperature during STEP 1B provides an increased number of electron/photon donors, hence lower ORP value (11.9) when the formulation is equilibrated (Day 2) when compared to H1A that was processed using STEP 1A. However, STEP 1B bounces back quickly during aging when compared to STEP 1A. The conclusion is that both STEP 1A and STEP 1B are adequate.

The frequency of 528 Hz (DNA repair) was imprinted to samples H2 and H4. The temperature that was used to imprint the frequencies plays a role in the shelf life. STEP 2A kept the frequency intact when compared to samples that only had their molecular structure processed (H1A and H1B) (STEP1 and STEP 1B) and were kept under shelf-life extension process STEP3. Interestingly, the frequency imprinting process also quickly increased the number of electron donors of the samples when samples H2 and H4 are equilibrated at Day 2, hence they had low ORP values (15 and 11 respectively) but they do not bounce back as high as sample H1B, wherein the ORP increase is more steady just like sample H1A. Sample H2 was successfully imprinted and the ORP results of the aged sample shows that the frequency is still present based on the comparison of samples H1A and H1B.

The frequency of 528 Hz (DNA repair) was imprinted to sample H3, but the sample was not conditioned with the same shelf-life extension process used for samples H2 and H4. The data clearly shows that the ORP values bounced back to a number similar to the sample G1/11 when aged.

Samples 14, 15, and 16 were imprinted using different timeframes (10, 20, 30 min, respectively) and the results were not as robust as prior tests, using the lab equipped PEMF device.

The table below shows results from Formula #2: Deuterium Depleted Water 40 ppm qs to 100%, Magnesium Sulfate @1%, Calcium Chloride@0.1%, Sodium Bicarbonate@0.1% and the ORP measurements after the addition of electron/photon enhancers after STEPS 1, 2 and 3 are completed. Formulas 3 and 4 are shown in the subsequent table. The table after shows the results when certain variations of the steps are performed.

| Formula #2 plus electron/photon | ORP | ORP Difference (mV) electron/photon |
|---|---|---|
| enhancers | mV | enhancers |
| CONTROL | 54 | |
| 1 Rosemary 1% | 61 | 7 |
| 2 Rhodochrosite 1% | 54 | 0 |
| 3 Hematite 1% | 34 | −20 |
| 4 Ginseng 1% | 44 | −10 |
| 5 HA as Sodium Hyaluronate | 21 | −33 |
| 0.5% | | |
| 6 St John's Wort 1% | 38 | −16 |
| 7 - xylitol complex void - became | | |
| gel | | |
| 8 Xylitol 0.7% | 45 | −9 |
| 9 methylene blue 0.05% | 30 | −24 |
| 10 Asthazanthin 0.18% | 55 | 1 |
| 11 Metalic Silver 0.25% | 57 | 3 |
| 12 CQ10 1% | 47 | −7 |
| 13 Diatomaceous Earth 0.5% | 34 | −20 |
| 14 Bentonite Clay 0.5% | 30 | −24 |
| 15 Snow Algae 0.2% | 32 | −22 |
| 16 Alantoin 0.25% | 54 | 0 |
| 17 Inulin 0.25% | 29 | −25 |
| HA 0.25% + St John's Wort | 37 | −17 |
| 0.5% | | |
| HA 0.15% + St John's Wort | 33 | −21 |
| 0.3% + methylene blue 0.015% | | |

Formulas #3 #4:

| | | | | | Formula 3 | Formula 4 |
|---|---|---|---|---|---|---|
| Phase | Functionality | Supplier | Trade Name | INCI | LULFS-001 | LULFS-002 |
| A | Solvent | Crystal | DI WATER | Water | 100.00 | 100.000 |
| A | Chelant | Evonik | Dermofeel PA | Phytic Acid (and) Aqua | 0.10 | 0.050 |
| A | pH adjuster | Spectrum | Citric Acid | Citric Acid | 0.10 | 0.050 |
| A | Buffer | Lotioncrafter | Sodium Citrate | Sodium Citrate | 0.15 | 0.075 |
| B premix | Thickener | BASF | Rheocare XGN | Xanthan Gum | 0.60 | 0.300 |
| B premix | Humectant | Genomatica | Brontide | Butylene Glycol | 5.00 | 2.500 |
| C | Humectant | Ajinomoto | Ajidew NL-50 | Sodium PCA (and) Aqua | 1.00 | 0.500 |
| C | Preservative | Schulke | Euxyl K 712 | Aqua (and) Sodium | 1.00 | 0.500 |
| | | | | Benzoate (and) | | |
| | | | | Potassium Sorbate | | |
| D | Electrolyte/Electron | Rona | Magnesium Sulfate | Magnesium Sulfate | 1.00 | 1.000 |
| | Enhancers | | | | | |
| D | Electrolyte/Electron | Milliard | Sodium Bicarbonate | Sodium Bicarbonate | 0.10 | 0.100 |
| | Enhancers | | | | | |
| D | Electrolyte/Electron | Modernist | Calcium Chloride | Calcium Chloride | 0.10 | 0.100 |
| | Enhancers | Pantry | | | | |
| E | Electron/Photon | Micro- | Sodium Hyaluronate | Sodium Hyaluronate | | 0.075 |
| | Enhancers | ingedients | | | | |
| E | Electron/Photon | Pure | St John's Wort | Water (and) Glycerin | | 0.150 |
| | Enhancers | Mountain | Extract | (and) Hypericum | | |
| | | Botanicals | | Perforatum Flower/ | | |
| | | | | Leaf/Stem Extract | | |
| E | Electron/Photon | Earth | Methylene Blue | Methylene Blue | | 0.008 |
| | Enhancers | Harmony | Pharmaceutical | | | |
| | | | Grade 99.99% with | | | |
| | | | with Liquid Gold | | | |

| | Description | | | | | | |
|---|---|---|---|---|---|---|---|
| | | | STEP 1A | STEP 1A | | | |
| | NO STEPS | STEP 1A | STEP 2B | STEP 2B | NO STEPS | STEP 2B | STEP 2B |
| | 1/2/3 | STEP 3 | STEP 3 | STEP 3 | 1/2/3 | STEP 3 | STEP 3 |
| | FORMULA 2 | FORMULA 2 | FORMULA 2 | FORMULA 2 | FORMULA3 | FORMULA 3 | FORMULA 4 |
| Sample IDs | J1 | J2 | J3′ | J3″ | LULFS-001 | LULFS-001 | LULFS-002 |
| Day 0 | 61 | 61 | 63 | 65 | 171 | 147 | 123 |
| Day 2 | 61 | 61 | 54 | 54 | | | |
| Difference | | | −9 | −11 | | | |
| (ORP) | | | | | | | |

Trials with formula 2 showed an increase in electrons in Sample J3 after the samples equilibrate (Day 2) when STEP 2 is completed.

The following conclusions can be drawn from the samples that are Formulas #3 and #4: when Formula #3 did not go through any STEPS, its ORP was 171, when STEP 2B (528 Hz frequency imprinting utilizing PEMF coil) and STEP 3 were performed, its ORP decreased to 147 mV.

When Formula #3 was injected with additional electron/photon enhancement ingredients becoming Formula #4, its ORP was reduced from 171 mV to 123 mV.

Studies were conducted using Spooky2 frequency generator, Spooky 2 PEMF coil, IKA hotplate, ORP Meter RCYAGO, laboratory room under wifi and blue visible light during experiments on formula 1 and under wifi and infrared light during experiments on formula 2, formula 3, and formula 4.

In an embodiment, the present invention relates to a topical preparation comprising components where an imprinted electromagnetic (EM) frequency, a combination of EM frequencies, and/or a quantum energy field frequenc(y)(ies), has/have been applied to the topical preparation for at least a first period of time.

In a variation, the electromagnetic (EM) frequenc(y)(ies) (or) the quantum energy field frequenc(y)(ies) are imprinted via a frequency generator. In a variation, the frequency generator generates a frequency comprising one or more members selected from the group consisting of wave shapes, harmonics, and duty cycles generated frequencies. In a variation, the EM frequenc(y)(ies) are applied to the topical preparation before use by an end user.

In a variation, the frequenc(y)(ies) is/are imprinted via a frequency generator in which the imprinted EM frequencies are in a range of 1-3000 Hz. In a variation, the EM frequenc(y)(ies) is/are imprinted via a frequency generator in which the imprinted EM frequenc(y)(ies) is/are in the range of 1-1000 Hz. In a variation, the EM frequenc(y)(ies) is/are imprinted via a frequency generator in which the imprinted frequencies are in the range of 10-1000 Hz.

In a variation, the imprinted electromagnetic (EM) frequency or combination of EM frequencies comprises a prolonged frequency duration. In a variation, the imprinted EM frequenc(y)(ies) in the topical preparation is/are boosted by a combination of natural ingredients that are rich in silicon and/or electrolytes. In a variation, the natural ingredients are one or more members selected from the group consisting of nicotinamide mononucleotide, peptides, CBD, melatonin, melanin, tyrosine, hematite, ginseng, sodium hyaluronate, hyaluronic acid, St. John's wort, xylitol, methylene blue, diatomaceous earth, coenzyme Q-10, bentonite clay, algae, allantoin, inulin, calcium chloride, magnesium sulfate, sodium bicarbonate, sodium chloride, and sucrose, or combinations thereof.

In an embodiment, the present invention relates toa topical preparation with imprinted EM frequenc(y)(ies) in a range of 1 Hz-3000 Hz, wherein the topical preparation further comprises a combination of one or more ingredients selected from the group consisting of nicotinamide mononucleotide, peptides, CBD, melatonin, melanin, tyrosine, hematite, ginseng, sodium hyaluronate, hyaluronic acid, St. John's wort, xylitol, methylene blue, diatomaceous earth, coenzyme Q-10, bentonite clay, algae, allantoin, inulin, calcium chloride, magnesium sulfate, sodium bicarbonate, sodium chloride, and sucrose, or combinations thereof wherein said one or more ingredients comprises that is delivered to and/or recognized by a user or a user's cells. In a variation, the information (or functionality) is delivered via native and/or non-native wavelengths that reach the user's cells. In a variation, the information that is delivered comprises quantum information. In a variation, the topical preparation further comprises one or more of tyrosine, arginine, melatonin, or retinoids, or combinations thereof. In a variation, the imprinted EM frequenc(y)(ies) comprise a combination of ingredients that aid in delivery of the information to a biofield of the user. In a variation, the topical preparation further comprises a combination of natural ingredients that are rich in silicon. In a variation, the topical preparation further comprises one or more natural ingredients comprising hematite, quartz, silver, gold, or diamond, or combinations thereof.

In an embodiment, the present invention relates to a method of preparing a topical preparation, the method comprising the steps of:
a) collecting one or more variables about a user of the topical preparation to generate a data set;
b) administering, via a processor, at least one artificial intelligence (AI) model to the data set to generate an AI driven data set;
c) administering, via a processor, at least one optimization engine to the AI driven data set to generate an optimized data set; and
d) generating, via a processor, the topical preparation based on the optimized data set.

In a variation, the method further includes a step of having an expert panel review the topical preparation to evaluate optimization of the topical preparation. In a variation, the topical preparation further comprises a companion product made by
a) collecting one or more variables about a user of the companion product to generate a data set;
b) administering, via a processor, at least one artificial intelligence (AI) model to the data set to generate an AI driven data set;
c) administering, via a processor, at least one optimization engine to the AI driven data set to generate an optimized data set; and
d) generating, via a processor, the companion product based on the optimized data set;
   wherein the topical preparation and the companion product is combined to generate a composition just prior to use, such as 30 seconds to 30 minutes prior to use.

In an embodiment, the present invention relates to a composition that comprises a topical preparation and a companion product. In a variation, the composition comprises about 90% to 99.9% by weight of the topical preparation; and about 0.1% to about 10% by weight of the companion product. In a variation, the topical preparation comprises a base formulation that comprises one or more of pH adjusting agents, one or more chelating agents, one or more emulsifiers, one or more emollients, one or more antimicrobial agents, one or more antioxidants, or one or more electrolytes, or combinations thereof. In a variation, the topical preparation may also contain a booster formulation. A booster formulation is also referred to as a companion product. In a variation, the topical preparation enhances the appearance or complexion of skin, hair, scalp, or fur, and optionally, also enhances the well-being of a user to which the topical preparation was applied.

In a variation, the composition enhances an appearance and/or a complexion of skin, hair, scalp, and/or fur, and optionally, and also optionally enhances a wellbeing of a user to which the topical preparation is applied.

In an embodiment, the user is a person or animal to which the topical preparation, the companion product or the composition is applied. In a variation, the user may be the person who is applying the topical preparation, the companion product or the composition. In a variation, the user may also be the person or animal that makes the composition, the topical preparation, and/or the companion product. In a variation, the user may be a person or animal about which data is collected to be used in the AI of the present invention.

Although this invention has been described with a certain degree of particularity, it is to be understood that the present disclosure has been made only by way of illustration and that numerous changes in the details of construction and arrangement of parts may be resorted to without departing from the spirit and the scope of the invention.

All references cited herein are incorporated by reference in their entireties for all purposes, including:
“Capone F, Dileone M, Profice P, Pilato F, Musumeci G, Minicuci G, Ranieri F, Cadossi R, Setti S, Tonali PA, Di Lazzaro V. Does exposure to extremely low frequency magnetic fields produce functional changes in human brain? J Neural Transm (Vienna). 2009 March.”
“Electric & Magnetic Fields.” National Institute of Environmental Health Sciences, https://www.niehs.nih.gov/health/topics/agents/emf. Accessed 26 May 2024.
“@inproceedings {Lee2014ConsensusRO, title={Consensus Recommendations on the Use of a Fractional Radiofrequency Microneedle and Its Applications in Dermatologic Laser Surgery}, author={Seung Jae Lee et al.”

What is claimed is:

1. A topical preparation comprising components where an imprinted electromagnetic (EM) frequency, a combination of EM frequencies, and/or (a) quantum energy field frequenc(y)(ies), has/have been applied to the topical preparation for at least a first period of time.

2. The topical preparation of claim 1 wherein the electromagnetic (EM) frequenc(y)(ies) (or) the quantum energy field frequenc(y)(ies) are imprinted via a frequency generator.

3. The topical preparation of claim 2, wherein the frequency generator generates a frequency comprising one or more members selected from the group consisting of wave shapes, harmonics, and duty cycles generated frequencies.

4. The topical preparation of claim 1 wherein the EM frequenc(y)(ies) are applied to the topical preparation before use by an end user.

5. The topical preparation of claim 1 wherein the frequenc(y)(ies) is/are imprinted via a frequency generator in which the imprinted EM frequencies are in a range of 1-3000 Hz.

6. The topical preparation of claim 1 wherein the EM frequenc(y)(ies) is/are imprinted via a frequency generator in which the imprinted EM frequenc(y)(ies) is/are in the range of 1-1000 Hz.

7. The topical preparation of claim 1 wherein the EM frequenc(y)(ies) is/are imprinted via a frequency generator in which the imprinted frequencies are in the range of 10-1000 Hz.

8. The topical preparation of claim 1, wherein the imprinted electromagnetic (EM) frequency or combination of EM frequencies comprises a prolonged frequency duration.

9. A topical preparation that comprises an imprinted EM frequenc(y)(ies) wherein the topical preparation is boosted by a combination of natural ingredients that are rich in silicon and/or electrolytes.

10. A topical preparation in claim 9, wherein the natural ingredients are one or more members selected from the group consisting of nicotinamide mononucleotide, peptides, CBD, melatonin, melanin, tyrosine, hematite, ginseng, sodium hyaluronate, hyaluronic acid, St. John's wort, xylitol, methylene blue, diatomaceous earth, coenzyme Q-10, bentonite clay, algae, allantoin, inulin, calcium chloride, magnesium sulfate, sodium bicarbonate, sodium chloride, and sucrose, or combinations thereof.

11. A topical preparation that comprises imprinted EM frequenc(y)(ies) in a range of 1 Hz-3000 Hz, wherein the topical preparation further comprises a combination of one or more ingredients selected from the group consisting of nicotinamide mononucleotide, peptides, CBD, melatonin, melanin, tyrosine, hematite, ginseng, sodium hyaluronate, hyaluronic acid, St. John's wort, xylitol, methylene blue, diatomaceous earth, coenzyme Q-10, bentonite clay, algae, allantoin, inulin, calcium chloride, magnesium sulfate, sodium bicarbonate, sodium chloride, and sucrose, or combinations thereof wherein said one or more ingredients comprises information that is delivered to and recognized by a user or a user's cells.

12. The topical preparation of claim 11, wherein the information is delivered via native and/or non-native wavelengths that reach the user's cells.

13. The topical preparation of claim 11, wherein the information that is delivered comprises quantum information.

14. The topical preparation of claim 11, wherein the topical preparation further comprises one or more of tyrosine, arginine, melatonin, or retinoids, or combinations thereof.

15. The topical preparation of claim 11, wherein the imprinted EM frequenc(y)(ies) comprise a combination of ingredients that aid in delivery of the information to a biofield of the user.

16. The topical preparation of claim 13 further comprises a combination of natural ingredients that are rich in silicon.

17. The topical preparation of claim 13 wherein the topical preparation further comprises one or more natural ingredients comprising hematite, quartz, silver, gold, or diamond, or combinations thereof.

18. A method of preparing a topical preparation, the method comprising the steps of:
a) collecting one or more variables about a user of the topical preparation to generate a data set;
e) administering, via a processor, at least one artificial intelligence (AI) model to the data set to generate an AI driven data set;
f) administering, via a processor, at least one optimization engine to the AI driven data set to generate an optimized data set; and
g) generating, via a processor, the topical preparation based on the optimized data set.

19. The method of claim 18, wherein the method further includes a step of having an expert panel review the topical preparation to evaluate optimization of the topical preparation.

20. The topical preparation of claim 1, further comprising a companion product made by
a) collecting one or more variables about a user of the companion product to generate a data set;
b) administering, via a processor, at least one artificial intelligence (AI) model to the data set to generate an AI driven data set;
c) administering, via a processor, at least one optimization engine to the AI driven data set to generate an optimized data set; and
d) generating, via a processor, the companion product based on the optimized data set;
wherein the topical preparation and the companion product is combined to generate a composition 30 seconds to 30 minutes prior to use.

21. The composition of claim 20, wherein the composition comprises about 90% to 99.9% by weight of the topical preparation; and about 0.1% to about 10% by weight of the companion product.

22. The topical preparation of claim 20 wherein the topical preparation comprises a base formulation that comprises one or more of pH adjusting agents, one or more chelating agents, one or more emulsifiers, one or more emollients, one or more antimicrobial agents, one or more antioxidants, or one or more electrolytes, or combinations thereof.

23. The topical preparation of claim 1, wherein the topical preparation enhances an appearance or complexion of skin, hair, scalp, or fur, and optionally, also enhances a wellbeing of a user to which the topical preparation was applied.

24. The composition of claim 20 wherein the composition enhances an appearance and/or a complexion of skin, hair, scalp, and/or fur, and optionally, and also optionally enhances a wellbeing of a user to which the topical preparation is applied.
